(19) United States
(12) Patent Application Publication
(10) Pub. No.: US 2025/0000479 A1
(43) Pub. Date: Jan. 2, 2025

(54) SYSTEM, METHOD AND COMPUTER READABLE MEDIUM FOR ANALYZING VASCULAR SOUND

(71) Applicant: Far Eastern Memorial Hospital, New Taipei City (TW)

(72) Inventors: I-No Cheng, New Taipei City (TW); Pen-Chih Liao, Taipei City (TW); Yen-Wen Wu, New Taipei City (TW); Yao-Chia Shih, Taipei City (TW); Shih-Hau Fang, Taoyuan City (TW)

(73) Assignee: Far Eastern Memorial Hospital, New Taipei City (TW)

(21) Appl. No.: 18/757,418

(22) Filed: Jun. 27, 2024

Related U.S. Application Data
(60) Provisional application No. 63/523,647, filed on Jun. 28, 2023.

(51) Int. Cl. A61B 7/02; A61B 5/00; A61B 5/02

(52) U.S. Cl. CPC A61B 7/02; A61B 5/02007; A61B 5/7264

(57) ABSTRACT

A system for analyzing vascular sound has a data acquiring module, a feature extraction module, and a feature analyzing module. The data acquiring module is used to acquire audio data from an individual. The feature extraction module is used to extract an audio feature from the audio data. The feature analyzing module is used to analyze the audio feature and to output an abnormality classification of a vascular sound corresponding to the audio data according to an analyze result of the audio feature.

### CROSS REFERENCE TO RELATED APPLICATIONS

This application claims the benefit of U.S. Provisional Application No. 63/523,647, filed on Jun. 28, 2023. The content of the application is incorporated herein by reference.

### BACKGROUND OF THE INVENTION

### 1. Field of the Invention

The present invention is related to medical diagnosis techniques, in particular to a system, a method, and a computer readable medium for analyzing vascular sound.

### 2. Description of the Prior Art

Patients with impaired kidney functionality require hemodialysis to maintain metabolism. Hemodialysis involves inserting a dialysis tube into the veins or arteries, leading blood out of the body to a hemodialysis machine to filter waste and excess water, then returning the blood. This can cause vascular access obstruction.

Hemodialysis typically requires arteriovenous access (AVA) in the forearm or upper arm. AVA can be autologous arteriovenous fistula (AVF) or arteriovenous graft (AVG). AVF uses the patient's vessels, while AVG uses artificial materials. Both types are prone to infection, blood clots, and stenosis, obstructing vascular access.

Therefore, a real-time solution is needed to determine AVA patency according to vascular sound and support clinical decisions.

### SUMMARY OF THE INVENTION

A system for analyzing vascular sound may include: a data acquisition module, a feature extraction module coupled with the data acquisition module, and a feature analysis module coupled with the feature extraction module. The data acquisition module may be used to acquire audio data from a subject in need thereof. The feature extraction module may be used to extract an audio feature from the audio data. The feature analysis module may be used to analyze the audio feature and output an abnormal classification corresponding to the audio data according to an analysis result of the audio feature.

A method for analyzing vascular sound may include: providing the system for analyzing vascular sound as mentioned above, and the data acquisition module acquiring the audio data of the subject.

A computer readable medium may store a computer executable instruction which, when executed, cause the method for analyzing vascular sound as mentioned above to be implemented.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of system for analyzing vascular sound.

FIG. 2 is a flow diagram of analysis method for analyzing vascular sound.

FIG. 3 is a flow diagram of model building method for building an artificial intelligence model.

FIG. 4 is an angiographic image of an arteriovenous access.

FIG. 5 is a block diagram of research process for building the artificial intelligence model using data of participated subjects.

FIG. 6 is a block diagram of acquiring ground truth dataset for building the artificial intelligence model.

FIG. 7 is a schematic diagram of a 30th second to 90th second segment of the 120-second audio data.

FIG. 8 is a block diagram of procedures for extracting audio feature from the audio data.

FIG. 9 is a schematic diagram of relationship between audio frequency and Mel scale.

FIG. 10A and FIG. 10B are schematic diagrams of Mel spectrum of the audio data obtained from procedures of FIG. 8.

FIG. 11A and FIG. 11B are schematic diagrams of representative MFCCs of the audio data obtained from procedures of FIG. 8.

FIG. 12A and FIG. 12B are schematic diagrams of power spectrums of the audio data for observing differences between AVA before PTA procedure and AVA after PTA procedure.

FIG. 13A and FIG. 13B are waveform diagrams of the audio data with respect to amplitude and time for observing differences between AVA before PTA procedure and AVA after PTA procedure.

FIG. 14A, FIG. 14B and FIG. 14C are confusion matrixes showing predicting performance of the artificial intelligence model built from CNN.

FIG. 15A, FIG. 15B and FIG. 15C are confusion matrixes showing predicting performance of the artificial intelligence model built from DNN.

FIG. 16 is a schematic diagram of ROC curves representing prediction efficiencies of CNN and DNN.

### DETAILED DESCRIPTION

The following describes the implementation of the present disclosure with examples. Those skilled in the art can easily understand the spirit, advantages and effects of the present disclosure from the content disclosed in this specification. However, the embodiments set forth herein are not intended to limit the present disclosure, and the present disclosure can also be implemented or applied by other different embodiments, and the details set forth herein can also be based on different viewpoints and applications. Various changes or modifications can be made without departing from the spirit of the present disclosure.

The features such as ratio, structure, and dimension shown in drawings accompanied with the present disclosure are provided to assist those skilled in the art to read and understand the present disclosure, rather than to limit the scope of implementation. Thus, in the case that does not affect the purpose of the present disclosure and the effect brought by the present disclosure, any change in proportional relationships, structural modification, or dimensional adjustment should fall within the scope of the technical contents disclosed herein. In addition, unless otherwise specified, the singular forms “a” and “the” used herein also include plural forms, and the terms “or” and “and/or” used herein are interchangeable.

When “comprising,” “including,” or “having” an element described herein, unless otherwise specified, other elements, components, structures, regions, parts, devices, systems, steps, or connection relationships and other requirements may be further included, rather than excluding those other requirements.

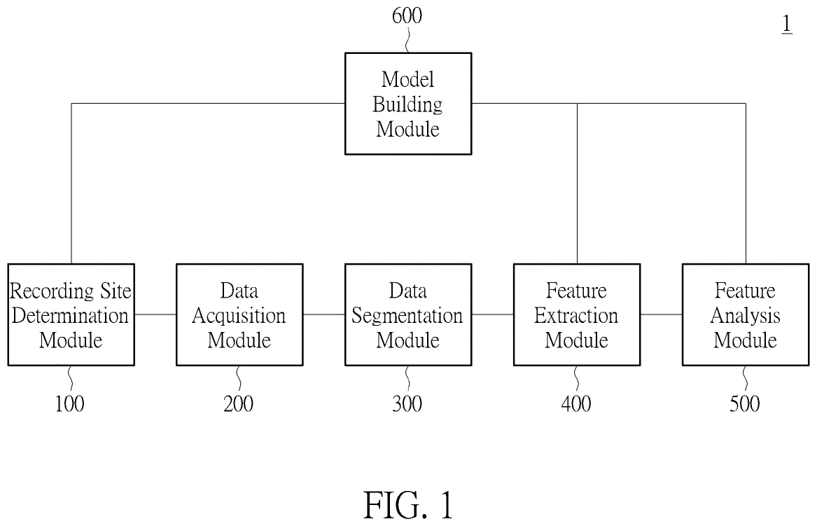

FIG. 1 is a schematic diagram of system 1 for analyzing vascular sound. The system 1 may include a recording site determination module 100, a data acquisition module 200, a data segmentation module 300, a feature extraction module 400, a feature analysis module 500 and a model building module 600. System 1 may enable personalized detection of abnormal vessel stenosis for subject in need of hemodialysis.

The recording site determination module 100 may be coupled to an angiography apparatus (not shown), and may be used to acquire angiography image of the subject and determine the recording site on body of the subject for determining abnormal stenosis of vessels. The recording site may be a position on body surface of the subject corresponding to a stenosis site of a vessel of the subject. In some embodiments, the vessel may be an arteriovenous access (AVA).

The data acquisition module 200 may be coupled to the recording site determination module 100 and an auscultation apparatus (not shown), and may be used to receive the audio data at the recording site of the subject through the auscultation device.

The data segmentation module 300 may be coupled to the data acquisition module 200, and may be used to segment audio data of the subject into segmented audio data corresponding to a predetermined length of time. The segmented audio data corresponding to the predetermined length of time may enable the system 1 to concentrate scope of analysis at specific audio features within the predetermined length of time of the segmented audio data. The predetermined length of time may correspond to length of time of at least one cardiac cycle of the subject.

The feature extraction module 400 may be coupled to the data segmentation module 300 and may be used to extract audio features from the segmented audio data corresponding to the predetermined length of time. The audio features may be Mel-frequency cepstral coefficients (MFCC) features, which may be extracted by Mel-frequency cepstrum technique performed by the feature extraction module 400.

The feature analysis module 500, coupled to the feature acquisition module 400, may use an artificial intelligence model to analyze MFCC features and output analysis results corresponding to an abnormal classification of the vascular sound indicated in the audio data. With the analysis result, a clinician may determine presence of abnormal stenosis at the stenosis site of the vessel of the subject, and then make a corresponding clinical decision to restore blood flow of the subject.

The model building module 600 may be coupled to the recording site determination module 100, the feature extraction module 400 and the feature analysis module 500, and may be used to build the artificial intelligence module of the feature analysis module 500 using the angiography image acquired from the recording site determination module 100 and the audio feature extracted from the feature extraction module 400.

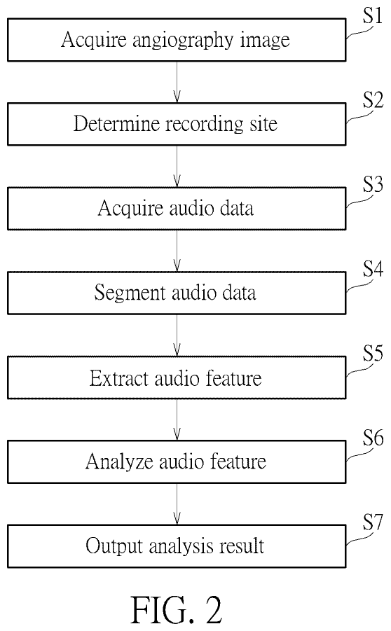

FIG. 2 is a flow diagram of analysis method 2 for analyzing vascular sound. The analysis method 2 may be performed by elements of the system 1, and may be used to determine abnormal stenosis of vessels of subject in need of hemodialysis in a personalized manner. The analysis method 2 may include the following steps:
S1: the recording site determination module 100 acquires the angiography image of the subject;
S2: the recording site determination module 100 determines the recording site on the subject according to the angiography image;
S3: the data acquisition module 200 acquires audio data from the recording site;
S4: the data segmentation module 300 segments the audio data into segmented audio data corresponding to the predetermined length of time;
S5: the feature extraction module 400 extracts the audio feature from the segmented audio data;
S6: the feature analysis module 500 analysis the audio feature; and
S7: the feature analysis module 500 outputs the analysis result of the audio feature.

In some embodiments, Step S1 and Step 2 of FIG. 2 may be omitted when the subject is a returning patient, the stenosis site of vessel of the subject is prone to recurrent abnormal stenosis and already known by the clinician, and audio data may be acquired directly from known recording site on the subject without assistance from the angiography image. In other embodiments, step S4 may be omitted when the data acquisition module 200 is capable of precisely acquiring the audio data according to one cardiac cycle, or the system 1 is configured to determine abnormal stenosis the vessel of the subject using the audio data without segmentation.

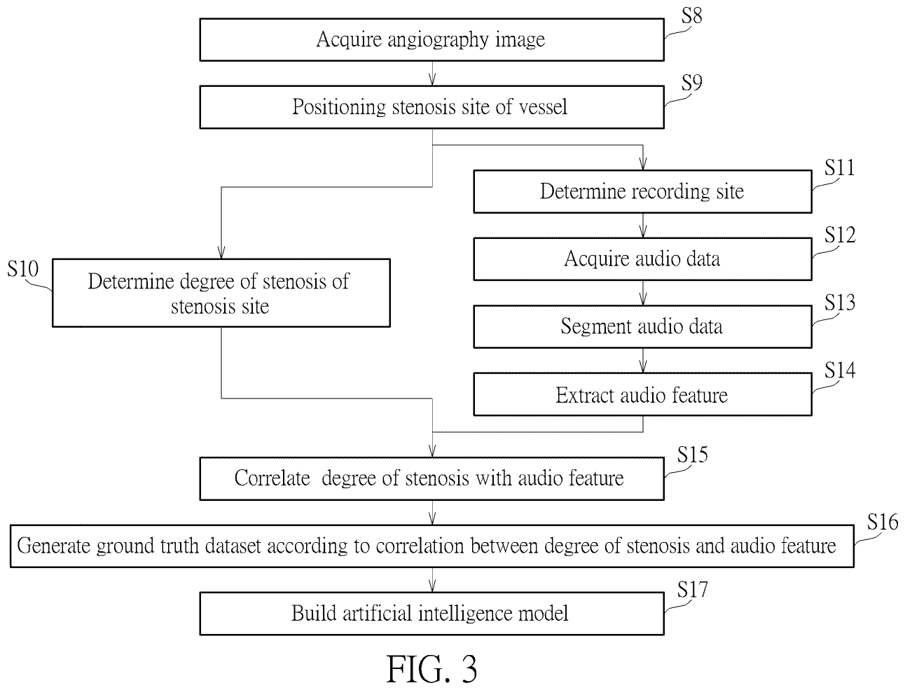

FIG. 3 is a flow diagram of model building method 3 for building the artificial intelligence model. The model building method 3 may be implemented in the model building module 600 and may include the following steps:
S8: the model building module 600 acquires the angiography image from the recording site determination module 100;
S9: the model building module 600 positions the stenosis site of the vessel from the angiography image;
S10: the model building module 600 determines degree of stenosis (DOS) of the stenosis site of the vessel according to the angiography image.
S11: the recording site determination module 100 determines the recording site on the subject according to the stenosis site;
S12: the data acquisition module 200 receives the audio data from the recording site;
S13: the data segmentation module 300 segments the audio data into the segmented audio data according to the predetermined length of time;
S14: the feature extraction module 400 extracts the audio feature from the segmented audio data;
S15: the model building module 600 acquires the audio feature and identifies a correlation between the audio feature and the DOS of the stenosis site of the vessel;
S16: the model building module 600 generates the ground truth dataset according to the correlation between the audio feature and the DOS;
S17: the model building module 600 builds the artificial intelligence model for the feature analysis module 500 according to the ground truth database.

In some embodiments, step 10 may be performed in parallel with Step S11 though Step S14. In other embodiments, step S13 may be omitted when the data acquisition module 200 is capable of precisely acquiring the audio data according to one cardiac cycle, or the system 1 is configured to determine abnormal stenosis the vessel of the subject using the audio data without segmentation.

In some other embodiments, a computer readable medium may be provided to store a computer executable instruction which, when being executed, causes the analysis method 2 and/or the model building model 3 to be implemented. The computer readable medium may be applied to a wearable apparatus or a mobile apparatus. A subject in need thereof may remotely access the system 1, the analysis method 2 and/or the model building method 3 via the wearable apparatus or the mobile apparatus having the computer readable medium to realize the analysis on vascular sounds and fulfill intent for telemedicine.

Below describes operational details of the system 1 and elements of the system 1, the analysis method 2, and the model building method 3.

### Research Motivation

### Types of Fistulas for Hemodialysis

The subject in need of hemodialysis treatment will first receive an arteriovenous fistula anastomosis on the forearm or upper arm to create an arteriovenous access (AVA) on the subject. AVA may connect high-pressure arteries to low-pressure veins. The AVA may be referred to as an autologous arteriovenous fistula (AVF) when the AVA is a vascular access made of tissue of the subject body and connects the artery and the vein. The AVA may be referred to as an arteriovenous graft (AVG) when the AVA is a vascular access made of biosynthetic material and connects the artery and the vein. The aim of the present invention is to instantly detect abnormal stenosis of AVF or AVG (hereinafter referred to as AVA) during the hemodialysis session of the subject, thereby to assist the clinician in making clinical decisions regarding the abnormal stenosis.

### Percutaneous Transluminal Angioplasty

Percutaneous Transluminal Angioplasty (PTA), also known as balloon dilation, is an interventional vascular surgery. When abnormal stenosis is presence at the AVA, the PTA may guide a balloon to the stenosis site having abnormal stenosis in the AVA through cardiac catheter technology, and inflate the balloon to remove clots or accumulations blocking the blood vessels. The aim of the present invention is to use data of the AVA of the subject from before and after the PTA as a detection criterion for abnormal stenosis.

### Diagnosis for Stenosis of Fistula

AVA is a vascular access that directly connects arteries and veins and is free of capillaries. The aim of the present invention is to determine presence of abnormal stenosis at the AVA by detecting the vascular sound emitted by the blood flowing through the AVA.

### Definition of DOS

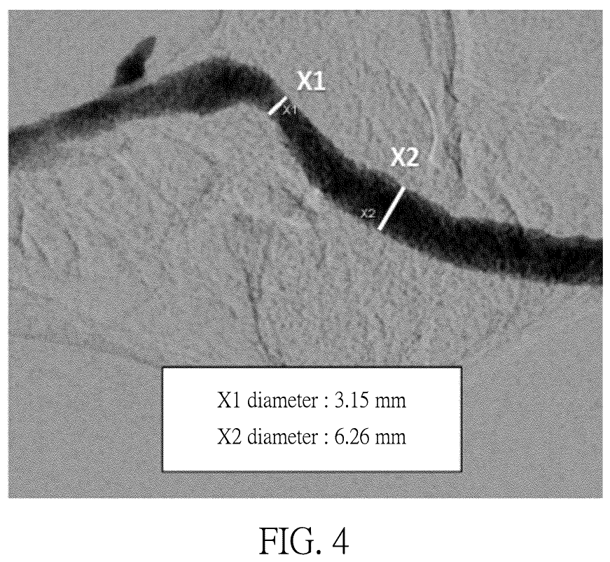

DOS represents the stenosis ratio of AVA relative to a reference diameter. FIG. 4 shows an angiographic image of an AVA marked with a reference diameter (marked as X2, which is about 6.26 mm wide) of a normal section of the AVA and a diameter (marked as X1, which is about 3.15 mm wide) of a stenosis site of the AVA experiencing abnormal stenosis. Therefore, DOS of X1 of stenosis site of the AVA relative to X2 of reference diameter of the AVA is calculated as 50%, which may be calculated through the following expression:

$$DOS \% = ( 1 - \frac{d}{D} ) \times 100 \% , \qquad expression ( 1 )$$

where d represents diameter of stenosis site of the AVA; and D represents reference diameter of the normal section of the AVA. When DOS of the stenosis site of the AVA is over 50% (i.e., DOS≥50%), the AVA is determined with presence of abnormal stenosis. When DOS of the stenosis site of the AVA is under 50% (i.e., DOS<50%), the AVA is determined to be free of abnormal stenosis.

Further, during determination for recording site of the subject while analysis method 2 or the model building method 3 is being performed, the stenosis site of the vessel may be determined using angiography image of the subject taken from current visit or previous visits to the clinic.

### Research Methodology

### Participants

The ground truth dataset for building the artificial intelligence model may relate to subjects undergoing long-term hemodialysis treatments through AVA and have experienced at least one PTA procedure. In the research, the demographic data of the subjects meeting the above condition is listed in Table 1 below:

TABLE 1
Demographic Data of Participants in Research

| Variable (Unit) | Value |
|---|---|
| Age (Year; Mean ± Standard Deviation) | 66.11 ± 11.91 |
| Sex | |
| Male (Percentage; Count) | 53.0%; 70 |
| Female (Percentage; Count) | 47.0%; 62 |
| Fistula Position | |
| Left Body (Percentage; Count) | 81.8%; 108 |
| Right Body (Percentage; Count) | 18.2%; 24 |
| Body Max Index (Kg/m²; Mean ± Standard Deviation) | 23.80 ± 3.78 |
| Risk Factor of Subject (Percentage; Count) | |
| Diabetes | 49.20%; 65 |
| Hypertension | 65.90%; 87 |
| Coronary Heart Disease | 27.30%; 36 |
| Stroke | 21.20%; 28 |
| Duration(Year; (min, max)) | 6.00; (2.00, 11.00) |
| Hemodialysis Age (Year; (min, max)) | 4.00; (2.00, 8.00) |
| Means for accepting Hemodialysis (Percentage; Count) | |
| Forearm AVF | 53.00%; 70 |
| Forearm AVG | 12.10%; 16 |
| Upper Arm AVF | 17.40%; 23 |
| Upper Arm AVG | 17.40%; 23 |
| AVF Total Count | 70.50%; 93 |
| AVG Total Count | 29.50%; 39 |
| AVA Diameter(mm; Mean ± Standard Deviation) | |
| Reference Diameter (Pre-PTA) | 6.98 ± 1.09 |
| Reference Diameter (Post-PTA) | 7.00 ± 1.04 |
| DOS of AVA (Percentage ± Standard Deviation) | |
| DOS (Pre-PTA) | 65.20% ± 13.81% |
| DOS (Post-PTA) | 28.05% ± 13.52% |
| Non-Invasive Blood Pressure Measurements (Mean ± Standard Deviation) | |
| Systolic | 159.16 ± 31.44 |
| Diastolic | 74.78 ± 13.49 |
| Mean Pressure | 102.91 ± 18.47 |
| Balloon Catheter (mm; Median(Q1, Q3)) | 7; (6, 7) |
| Heart Rate (Count/min) | 75.68 ± 11.72 |

Table 1 above listed 132 subjects being inducted in the research, without inducting subjects under age of 20, having diffused or multiple stenosis sites, having completely clogged AVA, and/or having stenosis site in AVA at unconventional positions that is hard to receive audio data. Moreover, the 132 subjects may be categorized into group AVF, group AVG, and group ALL AVA according to type of AVA on the subject. Group ALL AVA includes all 132 subjects regardless of them being in group AVF or group AVG.

### Research Process

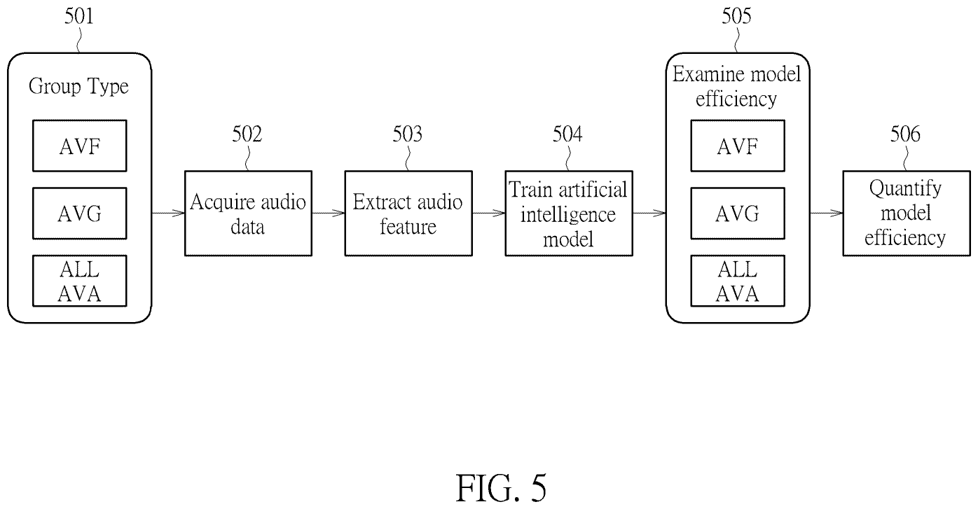

FIG. 5 is a block diagram of research process for building the artificial intelligence model using data of the 132 subjects. The research process may be used to verify result of the model building method 3 implemented by the model building module 600. At procedure 501, the subjects are categorized into group AVF, group AVG, and group ALL AVA according to type of the AVA on the subjects. At procedure 502, audio data is acquired for each of the subject; the audio data may correspond to vascular sound recorded from the AVA. At procedure 503, 26-dimensional MFCC feature is extracted from the vascular sound using Mel-frequency cepstrum technique. At procedure 504, the artificial intelligence model is trained to predict presence of abnormal stenosis at the AVA using the 26-dimensional MFCC feature. At procedure 505, the efficiency of the artificial intelligence model predicting presence of abnormal stenosis at the AVA for group AVA, group AVG and group ALL AVA are examined respectively. At procedure 506, the efficiency of the artificial intelligence model predicting presence of abnormal stenosis at the AVA for group AVA, group AVG and group ALL AVA are quantified, output as confusion matrix, and derived into indicators such as accuracy, sensitivity, specificity and F1-Score, respectively. Through procedures 501 to 506, the predicting performance of the artificial intelligence model may be evaluated.

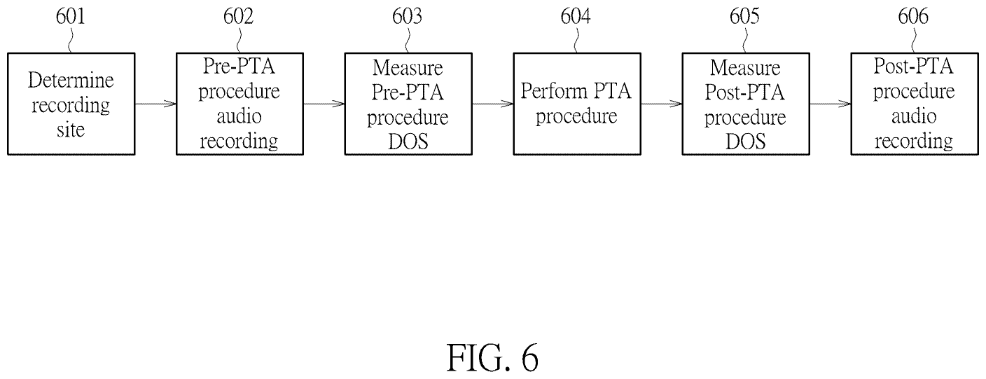

FIG. 6 is a block diagram of acquiring ground truth dataset for building the artificial intelligence model. The ground truth dataset may be used to confirm whether prediction result of the artificial intelligence model is conforming to actual stenosis condition of AVA of the subject. At procedure 601, recording site on the subject is determined at position corresponding to stenosis site prone to abnormal stenosis in the AVA; the stenosis site may be identified from the angiography image of the subject taken from current visit or previous visit to the clinic. At procedure 602, when presence of abnormal stenosis is confirmed and before the subject undergoes the PTA procedure, audio data of vascular sound at the stenosis site of the AVA is recorded via the recording site of the subject. At procedure 603, the DOS of the stenosis site of the AVA before the PTA procedure is determined using the angiography image of the subject before the PTA procedure. At procedure 604, the PTA procedure is performed on the subject with presence of abnormal stenosis in the AVA. At procedure 605, the DOS of the stenosis site of the AVA after the PTA procedure is determined using the angiography image of the subject after the PTA procedure. At procedure 606, after the subject undergoes the PTA procedure, audio data of vascular sound at the stenosis site of the AVA is recorded via the recording site of the subject.

The process illustrated in FIG. 6 may enable audio data and audio feature of the audio data of one subject before and after the PTA procedure to be correlated with DOS indicated in the angiography image of the subject and the clinical decision in response to the DOS made by clinicians for the subject, and hence formulate the ground truth dataset for the artificial intelligence model. Furthermore, even if subject during the current visit to the clinic is free of abnormal stenosis and not eligible for undergoing the PTA procedure, the audio data of the normal AVA of the subject may still be recorded, have audio features extracted therefrom, correlated (along with the audio features) with DOS indicated in the angiography image of the subject and the clinical decision in response to the DOS made by clinicians for the subject, and hence act as determination standard for predicting AVA without presence of abnormal stenosis.

During the research, each piece of audio data may include 120 seconds of recording, but only the segment from 11th second to 100th second of the audio data will be utilized to build the ground truth dataset and extract the 26-dimensional MFCC feature therefrom via the Mel-frequency cepstrum technique. The 90-second segment of the 120-second segment of the audio data has a relatively stable recording quality, and is more ideal for extraction of audio features and aiding prediction efficiency of the artificial intelligence model. Nevertheless, the 90-second segment of the 120-second segment of the audio data may be reconfigured to a longer or a shorter segment according to actual requirements, such as a 60-second segment, a 30-second segment or a 10-second segment. Furthermore, the 90-second segment of the 120-second segment of the audio data may be further segmented according to cardiac cycle of the subject to increase sample size of the ground truth dataset. That is, the artificial intelligence model may perform analysis on audio features for the audio data of each cardiac cycle of the same subject, thereby increasing the prediction efficiency of the artificial intelligence model.

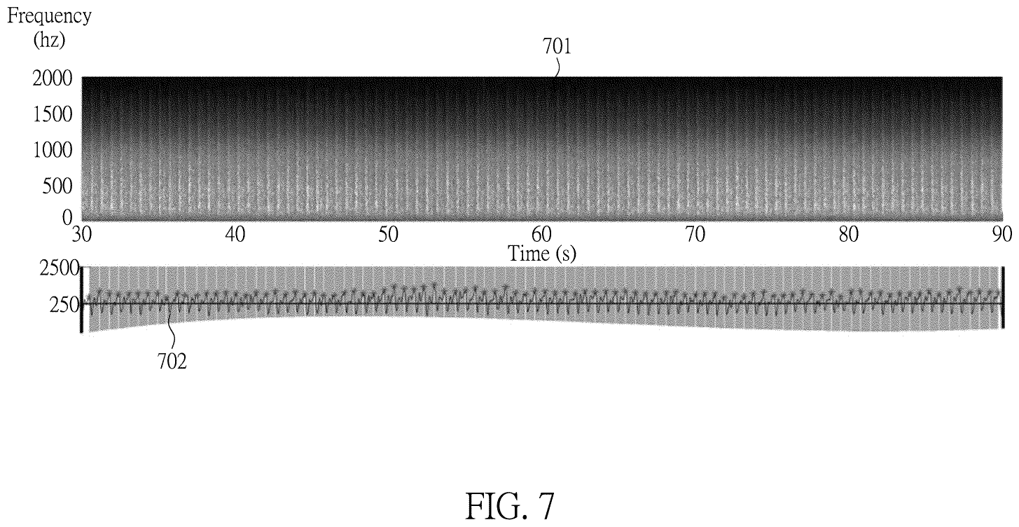

FIG. 7 is a schematic diagram of a 30th second to 90th second segment of the 120-second audio data. The audio data 701 may include frequency data corresponding to the vascular sound varying through time. The audio data 701 may be segmented according to cardiac cycles indicated in heartbeat data 702 of the subject to generate multiple segments of the audio data 701. These segments may enable the artificial intelligence model to concentrate scope of analysis at specific audio features during each cardiac cycle and process the audio data 701 in a much precise manner.

### Equipment for Experiment

The data acquisition module 200 may use an auscultation apparatus to record the audio data. The auscultation apparatus may be a multi-frequency positive tone electronic stethoscope, and may have an audio recording range between 20 Hz and 2000 Hz, a signal processing range up to 4000 Hz, and four channels for recording audio at the same time and individually adjusting audio recording gain. However, the auscultation apparatus is not meant to limit the scope of the present invention, and may be substituted with any suitable audio recording apparatus for obtaining the audio data.

### Feature Extraction

The Mel-frequency cepstrum technique applied for the feature extraction module 400 may be used to linearly transform the log energy spectrum of the nonlinear Mel scale of the audio frequency in the audio data, extract the audio features such as pitch differences, continuity, and volume of the vascular sound of the audio data, simulate perception manner of the human ear regarding hearing, and determine presence of DOS exceeding 50% in the AVA of the subject.

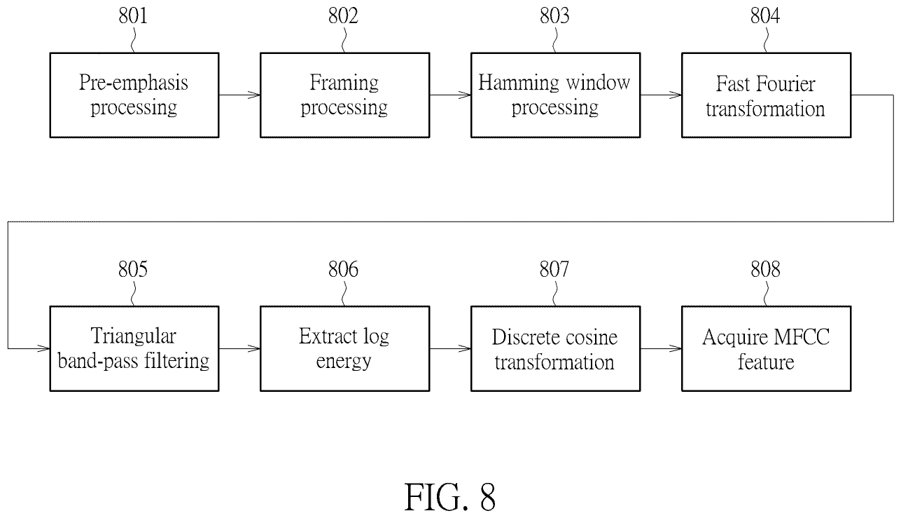

FIG. 8 is a block diagram of procedure 801 to procedure 808 for the feature extraction module 400 to extract the audio feature from the audio data.

At procedure 801, the audio data is passed through a high-pass filter to enhance energy at high-frequency portion of the signal of the audio data. The pre-emphasis processing in procedure 801 may increase signal-to-noise (noise-to-signal) ratio of the audio data, balance spectrum energy of the signal of the audio data, and reduce distortion in the audio data.

At procedure 802, the audio data is sampled into multiple frames for analysis. The sampling of frames is executed under assumption that the signal within each frame being stable. Further, the frames may be partially overlapped with each other to maintain continuity between adjacent frames.

At procedure 803, each frame from sampling is multiplied by the Hamming window to reduce discontinuity at edge of the frame and reduce sound leakage between frames.

At procedure 804, fast Fourier transform is performed on the frame to transform the frame from a time domain signal segment into a frequency domain signal segment, thereby to acquire energy spectrum of the frame. From here, energy intensity of the frame distributed in different frequency ranges may be observed in the frequency domain, spectral energy of the frame may be easily computed, and relevant audio features may be extracted and analyzed by analyzing the distribution of spectral energy.

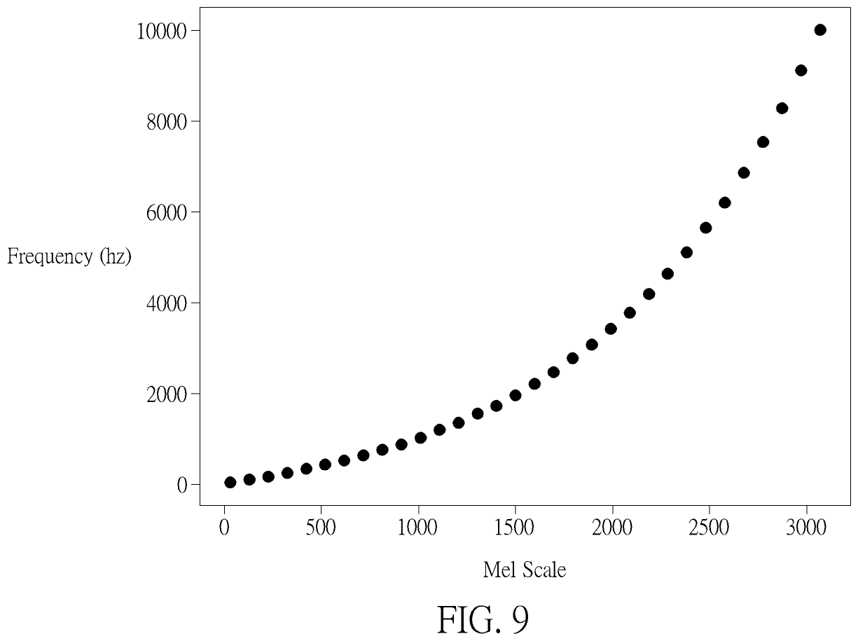

At procedure 805, the energy spectrum of each frame is passed through a triangular band-pass filter set to obtain a Mel spectrum corresponding to the energy spectrum. The triangular band-pass filter set may be evenly distributed on the Mel scale of the Mel spectrum. The Mel scale describes the nonlinear characteristics of the perception of human ear towards audio frequencies. The nonlinear characteristics are logarithmic changes according to relationship between audio frequency and the Mel scale shown in FIG. 9. The triangular band-pass filter may correspond to multiple triangular areas of the energy spectrum of the frame; the parameters and gain of the triangular band-pass filter may be adjusted to capture the audio features within the frame.

At procedure 806, the log energy of the frame is extracted from each Mel scale. From here, the spectral energy of the frame may be multiplied by the triangular band-pass filter to obtain the log energy output from each triangular band-pass filter. Extraction of the log energy may smooth out the spectrum of the frame, reduce data amount, and effectively capture the energy distribution of the frame. The sum of the log energy of each Mel scale represents the total energy of the signal intensity of the audio data.

At procedure 807, discrete cosine transformation is performed to transform the log energy of the frame into the cepstrum domain to obtain the cepstrum of the frame. Discrete cosine transformation may transform the log energy value of each triangular band-pass filter corresponding to the frame into a set of independent MFCCs. These MFCCs may include the audio features of the vascular sound of the audio data, and may be used for subsequent identification for abnormal stenosis.

At procedure 808, the MFCCs are output as audio features. From here, the output of audio features may include selecting 26 MFCCs related to amplitude of the ceptrum of the frame, which means the audio features will have feature vector of 26 dimensions.

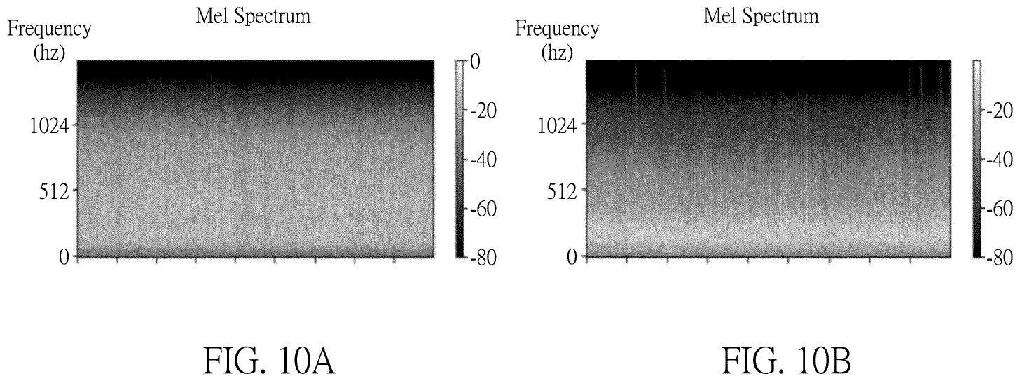

FIG. 10A and FIG. 10B are schematic diagrams of Mel spectrum of the audio data obtained from procedure 801 through procedure 808. FIG. 10A shows a Mel spectrum corresponding to vascular sound of an AVA with presence of abnormal stenosis; this Mel spectrum indicates a higher signal intensity at high-frequency band above 1000 Hz. FIG. 10B shows a Mel spectrum corresponding vascular sound of an AVA without presence of abnormal stenosis; this Mel spectrum indicates a higher signal intensity at frequency band below 500 Hz. Therefore, vascular sound with high frequency may be used as criteria to determine presence of abnormal stenosis.

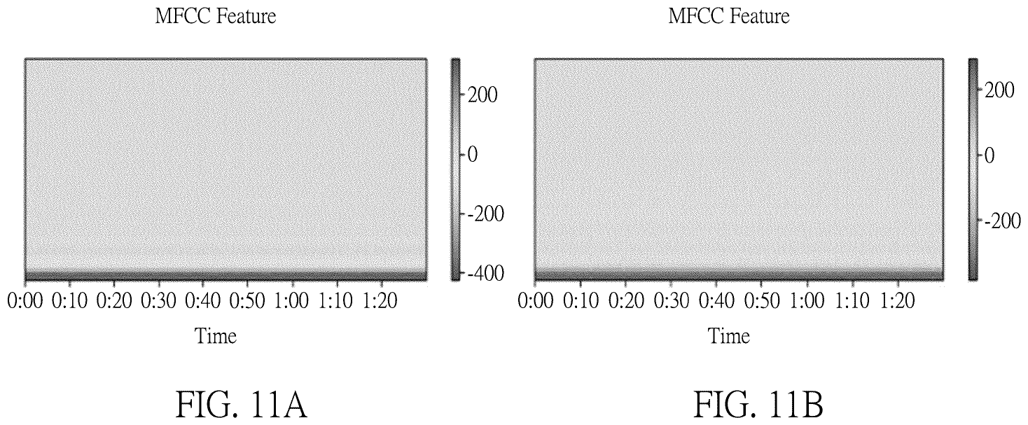

FIG. 11A and FIG. 11B are schematic diagrams of representative MFCCs of the audio data obtained from procedure 801 through procedure 808. In FIG. 11A and FIG. 11B, the Y axis represents the 26-dimensional MFCC feature, the X axis represents time, and the darker region represents a higher MFCC value within the range of time according to the X axis. In other words, FIG. 11A and FIG. 11B aims to visualize relationship between MFCCs and the audio data to express the following information:
I. Energy Distribution: energy distribution under different frequency ranges of the audio data may be observed through color variation presented in FIG. 11A and FIG. 11B. The darker region represents the frequency range with concentrated energy distribution; and the lighter region represents the frequency range with sparse energy distribution.
II. Frequency Characteristics: Audio features under different frequency ranges of the audio data may be observed through patterns of color variation presented in FIG. 11A and FIG. 11B. For example, spectacular or significant variation of energy within specific frequency range may be correlated to characteristics of the vascular sound of the audio data such as pitch, volume, resonance, etc.
III. Time Variation: Evolution of the audio data through time may be observed through color variation presented in FIG. 11A and FIG. 11B along the X axis. For example, appearance or disappearance of MFCC feature within specific range of time may represent appearance or disappearance of specific audio feature.

### Model Building Environment

The artificial intelligence model of the feature analysis module 500 may be based on a supervised learning architecture such as a convolutional neural network or a deep neural network. Each piece of data in the ground truth dataset for building the artificial intelligence model may be marked with at least one of the following attributes: AVA having DOS≥50%, AVA having DOS<50%, AVA in group AVF, AVA in group AVG, and/or AVA in group ALL AVA (regardless of group AVF or group AVG). The ground truth dataset may also be separated as the training set and the test set according to the ratio of 7:3 to train the artificial intelligence model.

### Convolutional Neural Network (CNN)

In some embodiments, the artificial intelligence model of the feature analysis module 500 based on CNN may include the architecture and related parameter settings listed in Table 2 below:

TABLE 2
examples of architecture and parameter settings of CNN

| Layers | Specification |
|---|---|
| Convolutional layer 1 | Filter × 4 (Size: 5 × 5; Stride: 1 × 1) |
| Pooling layer 1 | Size: 2 × 2 |
| Convolutional layer 2 | Filter × 16 (Size: 3 × 3; Stride: 1 × 1) |
| Pooling layer 2 | Size: 2 × 2 |
| Convolutional layer 3 | Filter × 32 (Size: 3 × 3; Stride: 1 × 1) |
| Convolutional layer 4 | Filter × 32 (Size: 3 × 3; Stride: 1 × 1) |
| Pooling layer 3 | Size: 2 × 2 |
| Batch normalization layer | Filter × 32 |
| Dropout layer 1 | Dropout rate: 0.25 |
| Flatten layer | |
| Dense layer 1 | Neurons × 256 |
| Dropout layer 2 | Dropout rate: 0.5 |
| Dense layer 2 | Neurons × 2 |
| Output layer | Softmax |

As listed in table 2, the convolution layer 1, the convolution layer 2, the convolution layer 3 and the convolution layer 4 may be realized as Conv2D two-dimensional convolution layer from Keras, which may extract MFCC features from the input MFCC diagram (such as those of FIG. 11A and FIG. 11B) in a two-dimensional sliding manner; the filters in each layer may extract MFCC features by performing convolution operations; the stride parameters set by the filters may set the output size of the convolution operations for each layer, where a larger stride may reduce the output size, and a smaller stride may increase the output size; the batch normalization layer may standardize and regularize the audio data to accelerate the model training process and improve model performance and stability; the dropout layer may randomly discard neurons in the CNN according to a predetermined discard rate, thereby reducing the dependence between neurons and enhancing the generalization ability of the CNN; the Softmax is an activation function to convert the original input of CNN into a probability distribution of possible abnormal classifications of vascular sound, where the sum of probabilities of all abnormal classifications is 1, and the abnormal classification with the maximum probability value may be output as prediction result of the abnormal classification of the vascular sound.

### Deep Neural Network (DNN)

In some embodiments, the artificial intelligence model of the feature analysis module 500 based on DNN may include the architecture and related parameter settings listed in Table 3 below:

TABLE 3
examples of architecture and parameter settings of DNN

| Layers | Specification |
|---|---|
| Dense layer 1 | Neurons × 128 |
| Batch normalization layer 1 | |
| Activation layer 1 | ReLU |
| Dropout layer 1 | Dropout rate: 0.4 |
| Dense layer 2 | Neurons × 128 |
| Batch normalization layer 2 | |
| Activation layer 2 | ReLU |
| Dropout layer 2 | Dropout rate: 0.5 |
| Dense layer 3 | Neurons × 128 |
| Batch normalization layer 3 | |
| Activation layer 3 | ReLU |
| Dropout layer 3 | Dropout rate: 0.5 |
| Dense layer 4 | Neurons × 2 |
| Output layer | Softmax |

As listed in table 3, each neuron in the dense layer 1, the dense layer 2, the dense layer 3 and the dense layer 4 is connected to all neurons in the previous layer; the batch normalization layer 1, the batch normalization layer 2 and the batch normalization 3 may perform batch normalization to accelerate training process of DNN and improve the generalization ability of DNN; the activation layer 1, the activation layer 2 and the activation layer 3 may utilize the linear rectification unit (ReLU) as the activation function to introduce nonlinearity transformation to change all negative values to zero and retain positive values; the dropout layer 1, the dropout layer 2 and the dropout layer 3 may randomly discard neurons in the CNN according to a predetermined discard rate, thereby reducing the dependence between neurons and prevent over-fitting due to over-reliance on specific neurons; the Softmax is an activation function to convert the original input of DNN into a probability distribution of possible abnormal classifications of vascular sound, where the sum of probabilities of all abnormal classifications is 1, and the abnormal classification with the maximum probability value may be output as prediction result of the abnormal classification of the vascular sound.

### Confusion Matrix

The invention may utilize the confusion matrix to reflect indicators such as accuracy, sensitivity, specificity, precision, negative predictive value (NPV), and F1-Score to evaluate the prediction efficiency of the artificial intelligence model. A confusion matrix may include the following four categories:
I. True Positive (TP): the amount of predictions identifying AVA with actual presence of abnormal stenosis to be AVA with presence of abnormal stenosis.
II. True Negative (TN): the amount of predictions identifying AVA without actual presence of abnormal stenosis to be AVA with presence of abnormal stenosis.
III: False Positive (FP): the amount of predictions identifying AVA without actual presence of abnormal stenosis to be AVA without presence of abnormal stenosis.
VI: False Negative (FN): the amount of predictions identifying AVA with actual presence of abnormal stenosis to be AVA without presence of abnormal stenosis.

Table 4 below presents the relationship between TP, TN, FP and FN in the confusion matrix and the prediction efficiency of the artificial intelligence model:

TABLE 4
Confusion Matrix

| | Actual Condition of AVA | | |
|---|---|---|---|
| | | With Abnormal Stenosis | Without Abnormal Stenosis |
| | | (DOS ≥ 50%) | (DOS < 50%) |
| Prediction | With Abnormal | TP | FP |
| Result | Stenosis | | |
| | Without Abnormal | FN | TN |
| | Stenosis | | |

The content of table 4 may be further utilized to compute indicators for prediction efficiencies of the artificial intelligence model:
I. Accuracy: accuracy describes the ratio of correct prediction by the artificial intelligence model using the ground truth dataset. Accuracy may be expressed as below:

$$Accuracy = \frac{TN + TP}{TN + TP + FN + FP} . \qquad expression ( 2 )$$

II. Sensitivity: sensitivity is also known as true positive rate (TPR) or recall rate, which describes the ability of the artificial intelligence model to correctly identify audio data corresponding to AVA with presence of abnormal stenosis from the ground truth dataset. Sensitivity may be expressed as below:

$$Sensitivity = \frac{TP}{TP + FN} . \qquad expression ( 3 )$$

III: Specificity: specificity is also known as true negative rate (TNR), which describes the ability of the artificial intelligence model to correctly identify audio data corresponding to AVA without presence of abnormal stenosis from the ground truth dataset. Specificity may be expressed as below:

$$Specificity = \frac{TN}{TN + FP} . \qquad expression ( 4 )$$

VI: Precision: precision describes correctness ratio of prediction by the artificial intelligence model to identify audio data corresponding to AVA with presence of abnormal stenosis. Precision may be expressed as below:

$$Precision = \frac{TP}{TP + FP} . \qquad expression ( 5 )$$

V: NPV: NPV describes correctness ration of prediction by the artificial intelligence model to identify audio data corresponding to AVA without presence of abnormal stenosis. NPV may be expressed as below:

$$NPV = \frac{TN}{TN + FN} . \qquad expression ( 6 )$$

VI F1-Score: F1-score describes prediction ability of the artificial intelligence models with respect to audio data corresponding to AVA with presence of abnormal stenosis. F1-score may be computed using sensitivity and precision, which is expressed as below:

$$F 1 - Score = \frac{2 \times ( Precision \times Sensitivity )}{( Precision + Sensitivity )} . \qquad expression ( 7 )$$

### Receiver Operating Characteristic Curve and Area Under Curve

The receiver operating characteristic curve (ROC curve) may be used to measure the performance of the artificial intelligence model in classifying AVA with presence of abnormal stenosis and AVA without presence of abnormal stenosis, where the vertical axis of the ROC curve represents sensitivity (true positive rate/recall rate), and the horizontal axis of the ROC curve represents false positive rate (FPR). The artificial intelligence model may be deemed as having a better efficiency when the ROC curve is close to the coordinate (0, 1). The false positive rate may be computed through the expression below:

$$FPR = \frac{FP}{FP + TN} . \qquad expression ( 8 )$$

Area Under Curve (AUC) refers to the area under the ROC curve, and may quantify the performance of the artificial intelligence model. The AUC ranges from 0 to 1 in value. The artificial intelligence model may be deemed as having a better performance when the AUC is bigger.

### Research Result

### Distribution of Audio Data

As described in FIG. 6, each of the participating 132 subjects in the research will undergo one recording before PTA procedure and one recording after PTA procedure. Therefore, the research will be conducted on total of 264 pieces of audio data, where each piece of the audio data will be recorded with 120 seconds of vascular sound of the AVA, and 90 seconds of recording within the 120-seconds audio data will be used for audio feature extraction. In other words, the 264 pieces of audio data may be denoted as table 5 below:

TABLE 5
Distribution of Audio Data

| DOS (%) | AVF | AVG | ALL AVA |
|---|---|---|---|
| ≥50% | 86 | 37 | 123 |
| <50% | 100 | 41 | 141 |
| Total Data Count | 186 | 78 | 264 |

### Frequency Distribution of Audio Data

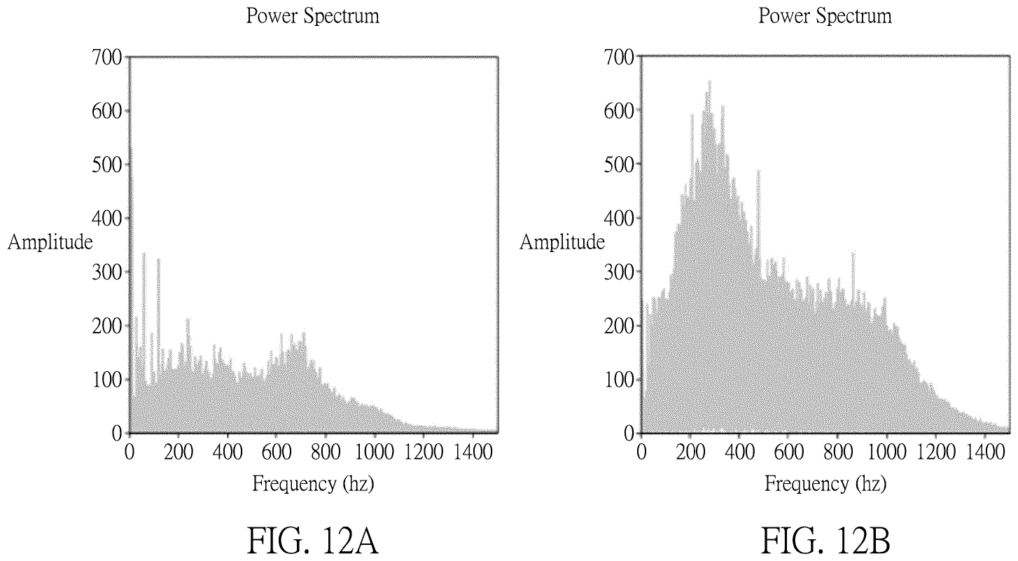

FIG. 12A and FIG. 12B are schematic diagrams of power spectrums of the audio data for observing differences between AVA before PTA procedure and AVA after PTA procedure. The power spectrum of FIG. 12A shows that vascular sound of the AVA has higher energy intensity in the high-frequency region above 600 Hz during presence of abnormal stenosis (in here DOS is 89%) within the AVA. The power spectrum of FIG. 12B shows that vascular sound of the AVA instead has higher energy intensity in the low-frequency region below 600 Hz after PTA procedure cleared out presence of abnormal stenosis (in here DOS has dropped to 26%) within the AVA.

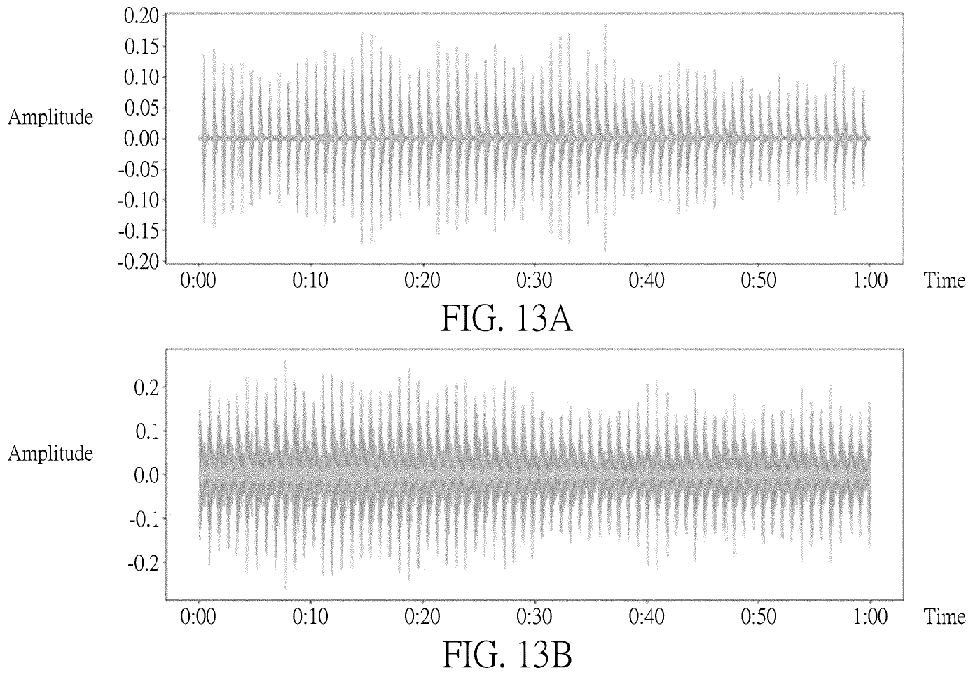

FIG. 13A and FIG. 13B are waveform diagrams of the audio data with respect to amplitude and time for observing differences between AVA before PTA procedure and AVA after PTA procedure. The waveform diagram of FIG. 13A shows presence of intermittent vascular sound through time since blood flow is severely clogged during presence abnormal stenosis (in here DOS is 90%) within the AVA. The waveform diagram of FIG. 13B shows presence of dense and continuous vascular sound during both systole and diastole of the heart after PTA procedure cleared out presence of abnormal stenosis (in here DOS is dropped to 35%) within the AVA.

### Prediction Result of Artificial Intelligence Model

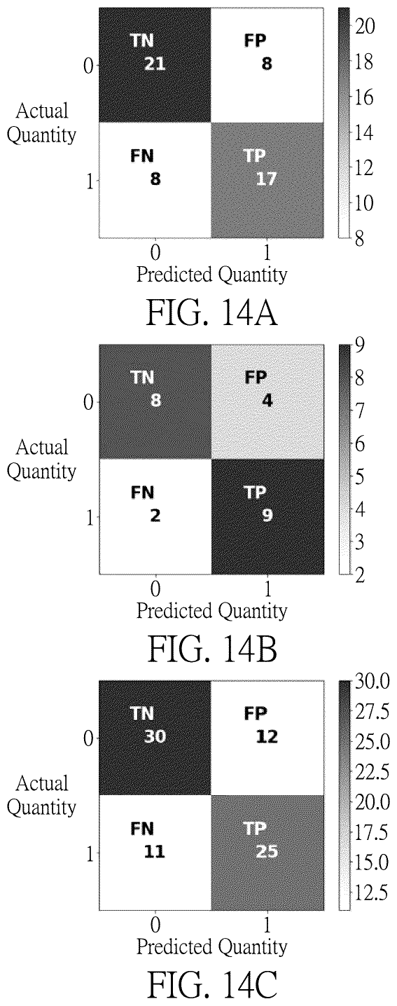

FIG. 14A, FIG. 14B and FIG. 14C are confusion matrixes showing predicting performance of the artificial intelligence model built from CNN using ground truth dataset of group AVF, group AVG and group ALL AVA respectively. From here, CNN has achieved the best sensitivity (81.82%) and F1-score (0.75) predicting presence of abnormal stenosis using ground truth dataset of group AVG with TP being 9 and FN being 2. In addition, CNN may also achieve at least 70% of accuracy predicting presence of abnormal stenosis judging from TP, FP, FN, and TN resulted from usage of ground truth dataset of group AVF, group AVG and group ALL AVA.

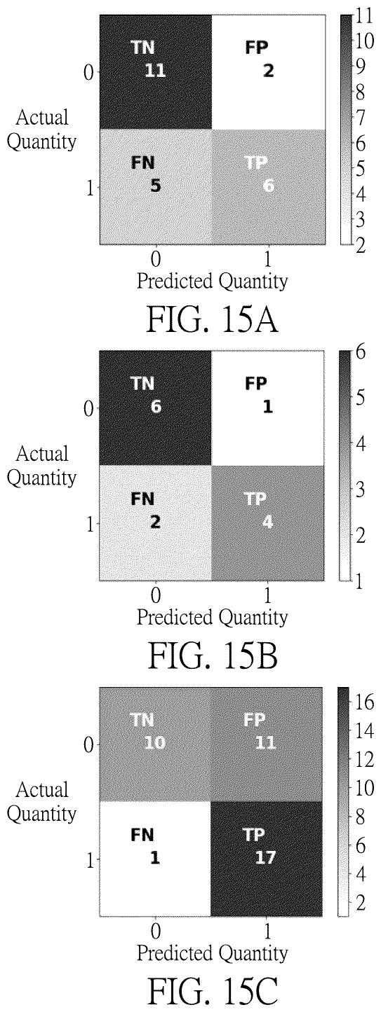

FIG. 15A, FIG. 15B and FIG. 15C are confusion matrixes showing predicting performance of the artificial intelligence model built from DNN using ground truth dataset of group AVF, group AVG and group ALL AVA respectively. From here, DNN has achieved relatively high specificity (86%), precision (80%), accuracy (77%), and F1-score (0.73) predicting presence of abnormal stenosis judging from TP, FP, FN, and TN resulted from usage of ground truth dataset of group AVG. In addition, DNN has achieved extreme high sensitivity (94%) predicting presence of abnormal stenosis judging from TP, FP, FN, and TN resulted from usage of ground truth dataset of group ALL AVA. Furthermore, DNN may also achieve at least 70% of accuracy predicting presence of abnormal stenosis judging from TP, FP, FN, and TN resulted from usage of ground truth dataset of group AVF, group AVG and group ALL AVA.

The research result from above also indicates that both CNN and DNN may achieve F1-score above 0.68 predicting presence of abnormal stenosis. The result proves that utilizing the artificial intelligence model for predicting presence of abnormal stenosis within AVA by using MFCC features of the vascular sound is a doable solution.

### ROC Curve and AUC

ROC curve and AUC of the prediction efficiency of CNN and DNN may be discussed according to content of FIG. 14A, FIG. 14B, FIG. 14C, FIG. 15A, FIG. 15B and FIG. 15C.

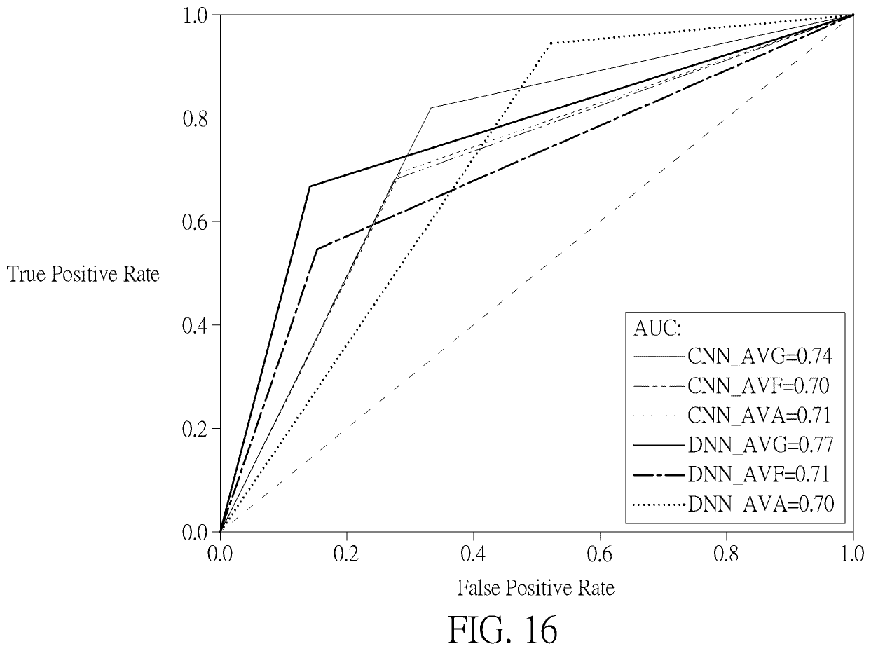

FIG. 16 is a schematic diagram of ROC curves representing prediction efficiencies of CNN and DNN using ground truth dataset of group AVF, group AVG and group ALL AVA respectively. From here, all ROC curves are shown to be close to coordinate (0, 1) on the top-left corner, and all AUCs corresponding to the ROC curves have values close to 1. Therefore, both CNN and DNN have excellent classification capability for audio feature of arbitrary AVA, and are applicable for predicting presence of abnormal stenosis within AVA.

### CONCLUSION

From the above, the system 1, analysis method 2 and model building method 3 of the present invention may achieve at least the three objectives below: I. establish ability of artificial intelligence model to identify presence of abnormal stenosis within AVA using Mel frequency cepstral techniques; II. directly identify condition of abnormal stenosis within arbitrary AVA using non-invasive digital auscultation approaches; and III. establish foundation for telemedicine using digital diagnostic approaches.

Those skilled in the art will readily observe that numerous modifications and alterations of the system, method, and computer readable medium may be made while retaining the teachings of the invention. Accordingly, the above disclosure should be construed as limited only by the metes and bounds of the appended claims.

What is claimed is:

1. A system for analyzing vascular sound, comprising:
a data acquisition module, configured to acquire audio data from a subject in need thereof;
a feature extraction module coupled with the data acquisition module, configured to extract an audio feature from the audio data; and
a feature analysis module coupled with the feature extraction module, configured to analyze the audio feature and output an abnormal classification corresponding to the audio data according to an analysis result of the audio feature.

2. The system of claim 1, wherein:
the audio data corresponds to the vascular sound of a vessel of the subject; and
the abnormal classification corresponds to degree of stenosis of the vessel indicated in the vascular sound.

3. The system of claim 2, wherein the vessel is an autologous arteriovenous fistula and/or an arteriovenous graft.

4. The system of claim 1, further comprising:
a data segmentation module coupling to the data acquisition module and the feature extraction module, configured to segment the audio data into a segmented audio data corresponding to a predetermined length of time, and transmit the segmented audio data corresponding to the predetermined length of time to the feature extraction module,
wherein the predetermined length of time corresponds to a length of time of at least one cardiac cycle of the subject.

5. The system of claim 1, wherein:
the audio feature is a Mel-frequency cepstral coefficient feature; and
the Mel-frequency cepstral coefficient feature is a 26-dimensional feature.

6. The system of claim 1, further comprising:
a recording site determination module coupled with the data acquisition module, configured to determine a recording site for acquiring the audio data of the subject according to an angiography image,
wherein the recording site is a position on the subject corresponding to a stenosis site of a vessel of the subject presented in the angiography image.

7. The system of claim 6, further comprising:
a model building module coupled with the recording site determination module, the feature extraction module, and the feature analysis module, configured to:
identify a correlation between the audio feature and degree of stenosis of the stenosis site presented in the angiography image;
generate a ground truth dataset according to the correlation; and
build an artificial intelligence model of the feature analysis module using the ground truth dataset.

8. The system of claim 7, wherein the artificial intelligence model comprises a convolutional neural network or a deep learning neural network.

9. A method for analyzing vascular sound, comprising:
providing the system for analyzing vascular sound of claim 1; and
the data acquisition module acquiring the audio data of the subject.

10. The method of claim 9, further comprising:
the feature extraction module extracting the audio data from the subject in need thereof;
the feature analysis module analyzing the audio feature; and
the feature analysis module outputting the abnormal classification corresponding to the audio data according to the analysis result of the audio feature.

11. The method of claim 10, wherein:
the audio data corresponds to the vascular sound of a vessel of the subject;
the abnormal classification corresponds to degree of stenosis of the vessel indicated in the vascular sound.

12. The method of claim 11, wherein the vessel is an autologous arteriovenous fistula and/or an arteriovenous graft.

13. The method of claim 10, further comprising:
a data segmentation module segmenting the audio data into a segmented audio data corresponding to a predetermined length of time; and
the data segmentation module transmitting the segmented audio data corresponding to the predetermined length of time to the feature extraction module,
wherein the predetermined length of time corresponds to a length of time of at least one cardiac cycle of the subject.

14. The method of claim 10, wherein:
the audio feature is a Mel-frequency cepstral coefficient feature; and
the Mel-frequency cepstral coefficient feature is a 26-dimensional feature.

15. The method of claim 10, further comprising:
a recording site determination module determining a recording site for acquiring the audio data of the subject according to an angiography image,
wherein the recording site is a position on the subject corresponding to a stenosis site of a vessel of the subject presented in the angiography image.

16. The method of claim 15, further comprising:
a model building module identifying a correlation between the audio feature and degree of stenosis of the stenosis site presented in the angiography image;
the model building module generating a ground truth dataset according to the correlation; and
the model building module building an artificial intelligence model of the feature analysis module using the ground truth dataset.

17. The method of claim 16, wherein the artificial intelligence model comprises a convolutional neural network or a deep learning neural network.

18. A computer readable medium storing a computer executable instruction which, when being executed, causes the method of claim 9 to be implemented.
